(19) United States
(12) Patent Application Publication
(10) Pub. No.: US 2008/0286845 A1
(43) Pub. Date: Nov. 20, 2008

(54) FERMENTATION PROCESS

(75) Inventor: Hans Sejr Olsen, Holte (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/115,084

(22) Filed: May 5, 2008

Related U.S. Application Data
(60) Provisional application No. 60/917,353, filed on May 11, 2007.

(30) Foreign Application Priority Data
May 8, 2007 (EP) 07107739.0

(51) Int. Cl. C12P 7/10

(52) U.S. Cl. 435/165; 435/161

(57) ABSTRACT

The present invention relates to processes of fermenting plant derived material into a desired fermentation product. The invention also relates to an antifoaming system for use in a fermentation process.

### CROSS-REFERENCE TO RELATED APPLICATIONS

This application claims priority or the benefit under 35 U.S.C. 119 of European application no. 07107739.0 filed May 8, 2007 and U.S. provisional application No. 60/917,353 filed May 11, 2007, the contents of which are fully incorporated herein by reference.

### FIELD OF THE INVENTION

The present invention relates to processes of fermenting plant derived material into a desired fermentation product. The invention also relates to an antifoaming system for use in a fermentation process.

### BACKGROUND OF THE INVENTION

A vast number of commercial products that are difficult to produce synthetically are today produced in fermentation processes. In such processes large amounts of foam may be formed. The foam lowers fermentation capacity per unit fermentor volume and may cause the fermenting liquid to overflow from the fermentor.

There is accordingly a demand for overcoming such a problem. An object of the present invention is therefore to provide an antifoaming system suitable for a fermentation process.

### SUMMARY OF THE INVENTION

In view of the foregoing circumstances, the present inventor has conducted an extensive investigation. As a result, it has been found that an antifoaming system for fermentation which is excellent in both foam-breaking effects and/or foam-inhibiting effects and/or does not adversely affect the fermentation production can be obtained by applying a lipolytic enzyme in combination with a metal salt.

Accordingly the invention provides in a first aspect a process for production of a fermentation product said process comprising contacting a fermentation media with a fermenting organism, a lipolytic enzyme and a metal salt.

In a second aspect the invention provides a composition suitable for use as an antifoaming system comprising a lipolytic enzyme and a metal salt. In a third aspect the invention provides use of such a composition in a fermentation process, e.g., in a process for production of ethanol.

According to the invention the lipolytic enzyme is preferably selected from the group consisting of phospholipase, lysophospholipase and lipase. Preferably the metal salt is selected from the group consisting of CaCl₂, CaCO₃, Ca(OH)₂, NaCl and KCl.

### DETAILED DESCRIPTION OF THE INVENTION

The antifoaming system of the present invention for fermentation can be applied to the fermentation of various substances. Application in anaerobic as well as aerobic fermentation is contemplated. It can be employed suitably for the fermentation of, for example, an amino acid, a carboxylic acid, an enzyme, an antibiotic, an alcohol or the like. Examples of preferred amino acids include glutamic acid, aspartic acid, citrulline, histidine, glutamine, isoleucine, leucine, lysine, ornithine, proline, serine, threonine, tryptophan and valine. The antifoaming system is particularly suited for the fermentation of glutamic acid and lysine. Examples of preferred carboxylic acids include citric acid, acetic acid, propionic acid, lactic acid, fumaric acid, tartaric acid, itaconic acid, alpha-ketoglutaric acid, ascorbic acid, gluconic acid, malic acid and kojic acid. Examples of preferred enzymes include alpha-amylase, beta-amylase, protease, lipase, cellulase, pectinase and glucoamylase. Examples of preferred antibiotic include beta-lactam antibiotics such as penicillin, aminoglucoside antibiotics such as kanamycin, chloramphenicol antibiotics, tetracycline antibiotics such as chlorotetracycline, macrolide antibiotics such as erythromycin, peptide antibiotics such as gramicidin S, antibacterial antibiotics such as mikamycin, novobiocin and lincomycin, antitumor antibiotics such as actinomycin D and chromomycin A3, and antifungal antibiotics such as azalomycin. Examples of preferred alcohols include ethanol, methanol, and butanol.

Although there is no particular limitation imposed on which fermentation process the antifoaming system of the present invention can be applied, the invention can be applied suitably to aerated culture, spinner culture, shaking culture or the like by which a large amount of foam is formed.

Preferably the fermentation process is a process for production of ethanol, and preferably the process includes fermentation with a yeast.

“Fermentation media” or “fermentation medium” refers to the environment in which the fermentation is carried out and which includes the fermentation substrate, that is, the carbohydrate source that is metabolized by the fermenting microorganism. The fermentation media, including fermentation substrate and other raw materials used in the fermentation process may be processed, e.g., by milling, liquefaction and saccharification processes or other desired processes prior to or simultaneously with the fermentation process. Accordingly, the fermentation media can refer to the media before the fermenting microorganisms are added, such as, the media in or resulting from a liquefaction or saccharification process, as well as the media which comprises the fermenting microorganisms, such as, the media used in a simultaneous saccharification and fermentation process (SSF). The carbohydrate source may be starch, e.g., such as provided by cereal grain, or it may be a cellulosic biomass, e.g., such as provided by corn stover or corn fiber, or any other suitable source of cellulosic matter.

“Fermenting microorganism” refers to any microorganism suitable for use in a desired fermentation process. Suitable fermenting microorganisms according to the invention are able to ferment, i.e., convert, sugars, such as glucose or maltose, directly or indirectly into the desired fermentation product. Examples of fermenting microorganisms include fungal organisms, such as yeast. Preferred yeast include strains of the Sacchromyces spp., and in particular, Sacchromyces cerevisiae. Commercially available yeast include, e.g., Red Star®/Lesaffre Ethanol Red (available from Red Star/Lesaffre, USA) FALI (available from Fleischmann's Yeast, a division of Burns Philp Food Inc., USA), SUPERSTART (available from Alltech), GERT STRAND (available from Gert Strand AB, Sweden) and FERMIOL (available from DSM Specialties).

### Lipolytic Enzymes

Preferred lipolytic enzymes for use in the antifoaming system of present invention are phospholipases (as classified by EC 3.1.1.4 and/or EC 3.1.1.32), lysophospholipases (as classified by EC 3.1.1.5) and lipases (as classified by EC 3.1.1.3, EC 3.1.1.23 and/or EC 3.1.1.26).

The lipolytic enzyme is preferably of microbial origin, in particular of bacterial, fungal or yeast origin. The lipolytic enzyme used may be derived from any source, including, for example, a strain of Absidia, in particular Absidia blakesleena and Absidia corymbifera, a strain of Achromobacter, in particular Achromobacter iophagus, a strain of Aeromonas, a strain of Alternaria, in particular Alternaria brassiciola, a strain of Aspergillus, in particular Aspergillus niger and Aspergillus flavus, a strain of Achromobacter, in particular Achromobacter iophagus, a strain of Aureobasidium, in particular Aureobasidium pullulans, a strain of Bacillus, in particular Bacillus pumilus, Bacillus strearothermophilus and Bacillus subtilis, a strain of Beauveria, a strain of Brochothrix, in particular Brochothrix thermosohata, a strain of Candida, in particular Candida cylindracea (Candida rugosa), Candida paralipolytica, and Candida antarctica, a strain of Chromobacter, in particular Chromobacter viscosum, a strain of Coprinus, in particular Coprinus cinerius, a strain of Fusarium, in particular Fusarium oxysporum, Fusarium solani, Fusarium solani pisi, and Fusarium roseum culmorum, a strain of Geotricum, in particular Geotricum penicillatum, a strain of Hansenula, in particular Hansenula anomala, a strain of Humicola, in particular Humicola brevispora, Humicola brevis var. thermoidea, and Humicola insolens, a strain of Hyphozyma, a strain of Lactobacillus, in particular Lactobacillus curvatus, a strain of Metarhizium, a strain of Mucor, a strain of Paecilomyces, a strain of Penicillium, in particular Penicillium cyclopium, Penicillium crustosum and Penicillium expansum, a strain of Pseudomonas, in particular Pseudomonas aeruginosa, Pseudomonas alcaligenes, Pseudomonas cepacia (syn. Burkholderia cepacia), Pseudomonas fluorescens, Pseudomonas fragi, Pseudomonas maltophilia, Pseudomonas mendocina, Pseudomonas mephitica lipolytica, Pseudomonas alcaligenes, Pseudomonas plantari, Pseudomonas pseudoalcaligenes, Pseudomonas putida, Pseudomonas stutzeri, and Pseudomonas wisconsinensis, a strain of Rhizoctonia, in particular Rhizoctonia solani, a strain of Rhizomucor, in particular Rhizomucor miehei, a strain of Rhizopus, in particular Rhizopus japonicus, Rhizopus microsporus and Rhizopus nodosus, a strain of Rhodosporidium, in particular Rhodosporidium toruloides, a strain of Rhodotorula, in particular Rhodotorula glutinis, a strain of Sporobolomyces, in particular Sporobolomyces shibatanus, a strain of Thermomyces, in particular Thermomyces lanuginosus (formerly Humicola lanuginosa), a strain of Thiarosporella, in particular Thiarosporella phaseolina, a strain of Trichoderma, in particular Trichoderma harzianum and Trichoderma reesei, and/or a strain of Verticillium.

In a preferred embodiment, the lipolytic enzyme used according to the invention is derived from a strain of Aspergillus, a strain of Achromobacter, a strain of Bacillus, a strain of Candida, a strain of Chromobacter, a strain of Fusarium, a strain of Humicola, a strain of Hyphozyma, a strain of Pseudomonas, a strain of Rhizomucor, a strain of Rhizopus, or a strain of Thermomyces.

In a preferred embodiment, the at least one lipolytic enzyme is a phospholipase. Phospholipases are enzymes which have activity towards phospholipids. Phospholipids, such as lecithin or phosphatidylcholine, consist of glycerol esterified with two fatty acids in an outer (sn-1) and the middle (sn-2) positions and esterified with phosphoric acid in the third position; the phosphoric acid, in turn, may be esterified to an amino-alcohol. Phospholipases are enzymes which participate in the hydrolysis of phospholipids. Several types of phospholipase activity can be distinguished, including phospholipases A₁ and A₂ which hydrolyze one fatty acyl group (in the sn-1 and sn-2 position, respectively) to form lysophospholipid; and lysophospholipase (or phospholipase B) which can hydrolyze the remaining fatty acyl group in lysophospholipid. Phospholipase C and phospholipase D (phosphodiesterases) release diacyl glycerol or phosphatidic acid respectively.

The term phospholipase includes enzymes with phospholipase activity, e.g., phospholipase A (A₁ or A₂), phospholipase B activity, phospholipase C activity or phospholipase D activity. The term “phospholipase A” used herein in connection with an enzyme of the invention is intended to cover an enzyme with Phospholipase A₁ and/or Phospholipase A₂ activity. The phospholipase activity may be provided by enzymes having other activities as well, such as, e.g., a lipase with phospholipase activity. The phospholipase activity may, e.g., be from a lipase with phospholipase side activity. In other embodiments of the invention the phospholipase enzyme activity is provided by an enzyme having essentially only phospholipase activity and wherein the phospholipase enzyme activity is not a side activity.

The phospholipase may be of any origin, e.g., of animal origin (such as, e.g., mammalian), e.g., from pancreas (e.g., bovine or porcine pancreas), or snake venom or bee venom. Alternatively, the phospholipase may be of microbial origin, e.g., from filamentous fungi, yeast or bacteria, such as the genus or species Aspergillus, e.g., A. niger, Dictyostelium, e.g., D. discoideum; Mucor, e.g., M. javanicus, M. mucedo, M. subtilissimus; Neurospora, e.g., N. crassa; Rhizomucor, e.g., R. pusillus; Rhizopus, e.g., R. arrhizus, R. japonicus, R. stolonifer, Sclerotinia, e.g., S. libertiana; Trichophyton, e.g., T. rubrum; Whetzelinia, e.g., W. sclerotiorum; Bacillus, e.g., B. megaterium, B. subtilis; Citrobacter, e.g., C. freundii; Enterobacter, e.g., E. aerogenes, E. cloacae; Edwardsiella, E. tarda; Erwinia, e.g., E. herbicola; Escherichia, e.g., E. coli; Klebsiella, e.g., K. pneumoniae; Proteus, e.g., P. vulgaris; Providencia, e.g., P. stuartii; Salmonella, e.g., S. typhimurium; Serratia, e.g., S. liquefasciens, S. marcescens; Shigella, e.g., S. flexneri; Streptomyces, e.g., S. violeceoruber, Yersinia, e.g., Y. enterocolitica. Thus, the phospholipase may be fungal, e.g., from the class Pyrenomycetes, such as the genus Fusarium, such as a strain of F. culmorum, F. heterosporum, F. solani, or a strain of F. oxysporum. The phospholipase may also be from a filamentous fungus strain within the genus Aspergillus, such as a strain of Aspergillus awamori, Aspergillus foetidus, Aspergillus japonicus, Aspergillus niger or Aspergillus oryzae. The phospholipase may also be from a filamentous fungus strain within the genus Thermomyces, such as Thermomyces lanuginosus (formerly Humicola lanuginosa). Preferred commercial phospholipases include LECITASE and LECITASE ULTRA (also known as HL1232) (available from Novozymes A/S). Suitable phospholipases are described in WO 98/26057 and WO 00/32758, which phospholipases are hereby incorporated by reference.

Phospholipases are preferably added in amounts from about 0.5 to 1000 LU/g DS in fermentation media, preferably, 1 to 400 LU/g DS, more preferably, 1 to 20 LU/g DS, such as, 1-10 LU/g DS.

In another preferred embodiment, the lipolytic enzyme is a lipase. Preferred lipases for use in the present invention included Candida antarcitca lipase and Candida cylindracea lipase. More preferred lipases are purified lipases such as Candida antarcitca lipase A, Candida antarcitca lipase B, Candida cylindracea lipase, and Penicillium camembertii lipase.

Preferred commercial lipases include LIPOLASE and LIPEX (available from Novozymes A/S) and G AMANO 50 (available from Amano).

Lipases are preferably added in amounts from about 0.5 to 1000 LU/g DS in fermentation media, preferably, 1 to 400 LU/g DS, more preferably 1 to 20 LU/g DS, such as, 1 to 10 LU/g DS and 1 to 5 LU/g DS.

In another preferred embodiment, combinations of lipolytic enzymes are used, such as (1) a lipase and a phospholipase, (2) a lipase and a lysophospholipase (3) a phospholipase and a lyso-phospholipase; and (4) a lipase, a phospholipase and a lyso-phospholipase.

### Additional Enzyme Activity

In a preferred embodiment, additional enzyme activity or activities may be used in combination with (such as prior to, during or following) the antifoaming system of the present invention. In addition to the enzymes traditionally used in starch processing, e.g., alpha-amylases and glucoamylases, preferred additional enzymes also include proteases, phytases, xylanases, cellulases, maltogenic alpha-amylases and beta-amylases.

Preferred alpha-amylases are of fungal or bacterial origin. More preferably, the alpha-amylase is a Bacillus alpha-amylase, such as, derived from a strain of B. licheniformis, B. amyloliquefaciens, and B. stearothermophilus. Other alpha-amylases include alpha-amylase derived from a strain of the Bacillus sp. NCIB 12289, NCIB 12512, NCIB 12513 or DSM 9375, all of which are described in detail in WO 95/26397, and the alpha-amylase described by Tsukamoto et al., 1988, Biochemical and Biophysical Research Communications 151: 2531. Other alpha-amylase variants and hybrids are described in WO 96/23874, WO 97/41213, and WO 99/19467. Other alpha-amylases include alpha-amylases derived from a strain of Aspergillus, such as, Aspergillus oryzae and Aspergillus niger alpha-amylases.

In a preferred embodiment, the alpha amylase is an acid alpha amylase. The term “acid alpha-amylase” means an alpha-amylase (E.C. 3.2.1.1) which when added in an effective amount has activity at a pH in the range of 3.0 to 7.0, preferably from 3.5 to 6.0, or more preferably from 4.0-5.0. Any suitable acid alpha-amylase may be used in the present invention.

In a preferred embodiment, the acid alpha-amylase is an acid fungal alpha-amylase or an acid bacterial alpha-amylase. Preferred acid alpha-amylase for use in the present invention may be derived from a strain of B. licheniformis, B. amyloliquefaciens, and B. stearothermophilus. More preferably, the acid alpha-amylase is acid fungal alpha amylases, such as, e.g., an acid alpha-amylase derived from Aspergillus niger.

Preferred commercial compositions comprising alpha-amylase include MYCOLASE (Gist Brocades), BAN™, TERMAMYL™ SC, FUNGAMYL™, LIQUOZYME™ X and SAN™ SUPER, SAN™ EXTRA L, NOVOZYM 50033 (Novozymes A/S) and CLARASE L-40,000, DEX-LO™, SPEYME FRED, SPEZYME™ AA, and SPEZYME™ M DELTA AA (Genencor Int.).

The alpha-amylase may be added in amounts as are well-known in the art. When measured in AAU units the acid alpha-amylase activity is preferably present in an amount of 5-500000 AAU/kg of DS, in an amount of 500-50000 AAU/kg of DS, or more preferably in an amount of 100-10000 AAU/kg of DS, such as 500-1000 AAU/kg DS. Fungal acid alpha-amylase are preferably added in an amount of 10-10000 AFAU/kg of DS, in an amount of 500-2500 AFAU/kg of DS, or more preferably in an amount of 100-1000 AFAU/kg of DS, such as approximately 500 AFAU/kg DS.

The glucoamylase may be derived from any suitable source, e.g., derived from a microorganism or a plant. Preferred glucoamylases are of fungal or bacterial origin, selected from the group consisting of Aspergillus glucoamylases, in particular A. niger G1 or G2 glucoamylase (Boel et al., 1984, EMBO J. 3(5): 1097-1102), or variants thereof, such as disclosed in WO 92/00381 and WO 00/04136; the A. awamori glucoamylase (WO 84/02921), A. oryzae (1991, Agric. Biol. Chem. 55(4): 941-949), or variants or fragments thereof.

Other Aspergillus glucoamylase variants include variants to enhance the thermal stability, such as, G137A and G139A (Chen et al., 1996, Prot. Engng. 9, 499-505); D257E and D293E/Q (Chen et al., 1995, Prot. Engng. 8: 575-582); N182 (Chen et al., 1994, Biochem. J. 301: 275-281); disulphide bonds, A246C (Fierobe et al., 1996, Biochemistry, 35: 8698-8704; and introduction of Pro residues in position A435 and S436 (Li et al., 1997, Protein Engng. 10: 1199-1204. Other glucoamylases include Talaromyces glucoamylases, in particular, derived from Talaromyces emersonii (WO 99/28448), Talaromyces leycettanus (U.S. Pat. No. RE 32,153), Talaromyces duponti, Talaromyces thermophilus (U.S. Pat. No. 4,587,215). Bacterial glucoamylases contemplated include glucoamylases from the genus Clostridium, in particular C. thermoamylolyticum (EP 135,138), and C. thermohydrosulfuricum (WO 86/01831).

Commercially available compositions comprising glucoamylase include AMG 200L; AMG 300 L; SAN™ SUPER, SAN™ EXTRA L, SPIRIZYME™ PLUS, SPIRIZYME™ FUEL and AMG™ E (from Novozymes A/S); AMIGASE™ and AMIGASE™ PLUS (from DSM); OPTIDEX™ 300, G-ZYME™ G900, G-ZYME™ and G990 ZR (from Genencor Int.).

Glucoamylases may in an embodiment be added in an amount of 0.02-2 AGU/g DS, preferably 0.1-1 AGU/g DS, such as 0.2 AGU/g DS.

In a preferred embodiment, the antifoaming system is used in combination with a phytase. In accordance with this embodiment, a phytase may be used, e.g., to promote the liberation of inorganic phosphate from phytic acid (myo-inositol hexakisphosphate) or from any salt thereof (phytates) present in the medium. The phytase may be added during the fermentation or prior to fermentation, such as, during propogation or in a step prior to fermentation, e.g., a liquefaction and/or saccharification step. The phytases made by added, e.g., to improve the bioavailability of essential minerals to yeast, as described in PCT Application WO 01/62947, which is hereby incorporated by reference.

In a preferred embodiment, the antifoaming system is used in combination with a protease. Proteases are well known in the art and refer to enzymes that catalyze the cleavage of peptide bonds. Suitable proteases include fungal and bacterial proteases. Preferred proteases are acidic proteases, i.e:, proteases characterized by the ability to hydrolyze proteins under acidic conditions below pH 7. Suitable acid fungal proteases include fungal proteases derived from Aspergillus, Mucor, Rhizopus, Candida, Coriolus, Endothia, Enthomophtra, Irpex, Penicillium, Sclerotium and Torulopsis. Especially contemplated are proteases derived from Aspergillus niger (see, e.g., Koaze et al., 1964, Agr. Biol. Chem. Japan, 28: 216), Aspergillus saitoi (see, e.g., Yoshida, 1954, J. Agr. Chem. Soc. Japan, 28: 66), Aspergillus awamori (Hayashida et al., 1977, Agric. Biol. Chem. 42(5): 927-933, Aspergillus aculeatus (WO 95/02044), or Aspergillus oryzae; and acidic proteases from Mucor pusillus or Mucor miehei.

Preferably, the protease is an aspartic acid protease, as described, for example, Handbook of Proteolytic Enzymes, Edited by A. J. Barrett, N. D. Rawlings and J. F. Woessner, Academic Press, San Diego, 1998, Chapter 270). Suitable examples of aspartic acid protease include, e.g., those disclosed in Berka et al., 1990, Gene 96: 313; (Berka et al., 1993, Gene 125: 195-198); and Gomi et al., 1993, Biosci. Biotech. Biochem. 57: 1095-1100, which are hereby incorporated by reference.

Suitable bacterial proteases include the commercially available products Alcalase® and Neutrase® (available from Novozymes A/S) and GC 106 and SPEZYME FAN (available from Genencor).

Protease may preferably be added in an amount of an amount of 10⁻⁷ to 10⁻⁵ gram active protease protein/g DS, in particular 10⁻⁷ to 5×10⁻⁶ gram active protease protein/g DS

In yet another preferred embodiment, the antifoaming system is used in combination with a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. Examples of maltogenic alpha-amylases include the maltogenic alpha-amylase from B. stearothermophilus strain NCIB 11837. Maltogenic alpha-amylases are described in U.S. Pat. Nos. 4,598,048, 4,604,355 and 6,162,628, which are hereby incorporated by reference. A commercially available maltogenic amylase is MALTOGENASE™ (available from Novozymes A/S). Preferably, the maltogenic alpha-amylase is used in a raw starch hydrolysis process to aid the formation of retrograded starch. Preferably, the lipolytic enzyme is combined with the maltogenic alpha-amylase in a liquefaction process. Preferably, the maltogenic alpha-amylase is added in an amount of 0.02 to 1.0 g/DS.

In yet another preferred embodiment, the antifoaming system is used in combination with a beta-amylase. Beta-amylase (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (Fogarty and Kelly, 1979, Progress in Industrial Microbiology 15: 112-115). These beta-amylases are characterized by having optimum temperatures in the range from 40° C. to 65° C. and optimum pH in the range from 4.5 to 7. Other examples of beta-amylase include the beta-amylases described in U.S. Pat. No. 5,688,684. Commercially available beta-amylases include NOVOZYM WBA® (from Novozymes A/S) and SPEZYME™ BBA 1500 and OPTIMALT (from Genencor Int., USA).

In another preferred embodiment, the antifoaming system is used in combination with an xylanase. The xylanase (E.C. 3.2.1.8) activity may be derived from any suitable source, including fungal and bacterial organisms, such as Aspergillus, Disporotrichum, Penicillium, Neurospora, Fusarium and Trichoderma.

In yet another preferred embodiment, the antifoaming system is used in combination with a cellulase. The cellulase activity used according to the invention may be derived from any suitable origin, preferably, the cellulase is of microbial origin, such as derivable from a strain of a filamentous fungus (e.g., Aspergillus, Trichoderma, Humicola, Fusarium). Cellulases are preferably applied in embodiments comprising enzymatic hydrolysis of cellulosic biomass.

The term “cellulases” as used herein are understood as comprising the cellobiohydrolases (EC 3.2.1.91), e.g., cellobiohydrolase I and cellobiohydrolase II, as well as the endoglucanases (EC 3.2.1.4).

In order to be efficient, the digestion of cellulose requires several types of enzymes acting cooperatively. At least three categories of enzymes are necessary to convert cellulose into glucose: endoglucanases (EC 3.2.1.4) that cut the cellulose chains at random; cellobiohydrolases (EC 3.2.1.91) which cleave cellobiosyl units from the cellulose chain ends and beta-glucosidases (EC 3.2.1.21) that convert cellobiose and soluble cellodextrins into glucose. Among these three categories of enzymes involved in the biodegradation of cellulose, cellobiohydrolases are the key enzymes for the degradation of native crystalline cellulose. The term “cellobiohydrolase I” is defined herein as a cellulose 1,4-beta-cellobiosidase (also referred to as exo-glucanase, exo-cellobiohydrolase or 1,4-beta-cellobiohydrolase) activity, as defined in the enzyme class EC 3.2.1.91, which catalyzes the hydrolysis of 1,4-beta-D-glucosidic linkages in cellulose and cellotetraose, by the release of cellobiose from the non-reducing ends of the chains. The definition of the term “cellobiohydrolase II activity” is identical, except that cellobiohydrolase II attacks from the reducing ends of the chains.

Endoglucanases (EC No. 3.2.1.4) catalyses endo hydrolysis of 1,4-beta-D-glycosidic linkages in cellulose, cellulose derivatives (such as carboxy methyl cellulose and hydroxy ethyl cellulose), lichenin, beta-1,4 bonds in mixed beta-1,3 glucans such as cereal beta-D-glucans or xyloglucans and other plant material containing cellulosic parts. The authorized name is endo-1,4-beta-D-glucan 4-glucano hydrolase, but the abbreviated term endoglucanase is used in the present specification.

The cellulolytic activity may, in a preferred embodiment, be derived from a fungal source, such as a strain of the genus Trichoderma, preferably a strain of Trichoderma reesei; or a strain of the genus Humicola, such as a strain of Humicola insolens.

Commercially available preparations comprising cellulase which may be used include CELLUCLAST®, CELLUZYME®, CEREFLO® and ULTRAFLO® (Novozymes A/S), LAMINEX™ and SPEZYME® CP (Genencor Int.) and ROHAMENT® 7069 W (from Röhm GmbH).

The enzymes applied in the antifoaming system of the present invention may be derived or obtained from any suitable origin, including, bacterial, fungal, yeast or mammalian origin. The term “derived” or means in this context that the enzyme may have been isolated from an organism where it is present natively, i.e., the identity of the amino acid sequence of the enzyme are identical to a native enzyme. The term “derived” also means that the enzymes may have been produced recombinantly in a host organism, the recombinant produced enzyme having either an identity identical to a native enzyme or having a modified amino acid sequence, e.g., having one or more amino acids which are deleted, inserted and/or substituted, i.e., a recombinantly produced enzyme which is a mutant and/or a fragment of a native amino acid sequence or an enzyme produced by nucleic acid shuffling processes known in the art. Within the meaning of a native enzyme are included natural variants. Furthermore, the term “derived” includes enzymes produced synthetically by, e.g., peptide synthesis. The term “derived” also encompasses enzymes which have been modified e.g., by glycosylation, phosphorylation, or by other chemical modification, whether in vivo or in vitro. The term “obtained” in this context means that the enzyme has an amino acid sequence identical to a native enzyme. The term encompasses an enzyme that has been isolated from an organism where it is present natively, or one in which it has been expressed recombinantly in the same type of organism or another, or enzymes produced synthetically by, e.g., peptide synthesis. With respect to recombinantly produced enzymes the terms “obtained” and “derived” refers to the identity of the enzyme and not the identity of the host organism in which it is produced recombinantly.

The enzymes may also be purified. The term “purified” as used herein covers enzymes free from other components from the organism from which it is derived. The term “purified” also covers enzymes free from components from the native organism from which it is obtained. The enzymes may be purified, with only minor amounts of other proteins being present. The expression “other proteins” relate in particular to other enzymes. The term “purified” as used herein also refers to removal of other components, particularly other proteins and most particularly other enzymes present in the cell of origin of the enzyme of the invention. The enzyme may be “substantially pure,” that is, free from other components from the organism in which it is produced, that is, for example, a host organism for recombinantly produced enzymes. In preferred embodiment, the enzymes are at least 75% (w/w) pure, more preferably at least 80%, at least 85%, at least 90%, at least 95%, at least 96%, at least 97%, at least 98%, or at least 99% pure. In another preferred embodiment, the enzyme is 100% pure.

### Metal Salts

Any suitable metal salt may be applied in the antifoaming system of the present invention. Preferred metal salts include salts of a metal selected from the group consisting of Ca, Mg, Na, K. More preferred metal salts includes salts selected from the group consisting of CaCl₂, CaCO₃, Ca(OH)₂, NaCl and KCl. Most preferred metal salts are salts of divalent metal ions, such as CaCl₂, CaCO₃ and Ca(OH)₂.

### Processes

The antifoaming system described herein are preferably used in combination with a fermentation processes. Fermentation processes are well known in the art. A fermentation process usually includes liquefaction or saccharification of a raw material comprising starch, e.g., from grain. Any liquefaction or saccharification may be used in combination with the fermentation process of the present invention. According to the present invention, the saccharification and liquefaction may be carried out simultaneously or separately with the fermentation process. In a preferred embodiment of the present invention, the liquefaction, saccharification and fermentation processes are carried out simultaneously.

The raw material for the fermentation processes may in particular be obtained from tubers, roots, stems, cobs, legumes, cereals or whole grain. More specifically the granular starch may be obtained from corns, cobs, wheat, barley, rye, milo, sago, cassava, tapioca, sorghum, rice, peas, bean, banana or potatoes. Preferred are both waxy and non-waxy types of corn and barley.

In the production of fermentation products, e.g., ethanol and other starch-based products, the raw material, such as whole grain, preferably corn, is milled in order to open up the structure and allow for further processing. Two processes are preferred according to the invention: wet milling and dry milling. Preferred is dry milling where the whole grain is milled and used in the remaining part of the process. Wet milling may also be used and gives a good separation of germ and meal (starch granules and protein) and is with a few exceptions applied at locations where there is a parallel production of syrups. Both wet and dry milling processes are well known in the art.

The antifoaming system described herein is suitable for application in fermentation processes comprising thermal gelatinization of the milled grain (“traditional fermentation processes”) as well as in fermentation processes which does not comprise such a thermal gelatinization (“raw starch hydrolysis and fermentation processes” or “RSH”). Traditional fermentation processes wherein the antifoaming system of the present invention may be applied are described in WO 1996/28567 and WO 2002/38787, all of which are hereby incorporated by reference. RSH processes wherein the antifoaming system of the present invention may be applied are described in U.S. Pat. No. 4,316,956, WO 2003/66816, WO 2003/66826 and WO 2004/080923, all of which are hereby incorporated by reference. Furthermore, the antifoaming system described herein is suitable for application in fermentation processes comprising enzymatic and/or acid hydrolysis of biomass preferably furthermore comprising to fermentation, e.g., to ethanol, such as described in US 2006/110891, US 2006/110900, WO 2005/100582, WO 2006/125068 and WO 2006/101832, all of which are hereby incorporated by reference. Any material comprising plant cell wall polysaccharides, e.g., wood, agricultural residues, herbaceous crops, and municipal solid wastes can be used as sources of biomass.

A traditional fermentation process typically includes thermal gelatinization of the granular starch as part of the liquefaction step. “Liquefaction” is a step in which milled (whole) grain raw material is broken down (hydrolyzed) into maltodextrins (dextrins). The liquefaction step is typically carried out using an alpha-amylase. Liquefaction is often carried out as a three-step hot slurry process. The slurry is heated to between 60-95° C., preferably 80-85° C., and the enzymes are added to initiate liquefaction (thinning). The slurry is then jet-cooked at a temperature between 95-140° C., preferably 105-125° C. to complete gelatinization of the slurry. Then the slurry is cooled to 60-95° C. and more enzyme(s) is(are) added to finalize hydrolysis (secondary liquefaction). The liquefaction process is usually carried out at pH 4.5-6.5, in particular at a pH between 5 and 6.

“Saccharification” is a step in which the maltodextrin (such as, produced from the liquefaction process) is converted to low molecular sugars DP₁₋₃ (i.e., carbohydrate source) that can be metabolized by the fermenting organism, such as, yeast. Saccharification processes are well known in the art and are typically performed enzymatically using a glucoamylase. Alternatively or in addition, alpha-glucosidases or acid alpha-amylases may be used. A full saccharification step may last up to from about 24 to about 72 hours, and is often carried out at temperatures from about 30 to 65° C., and at a pH between 4 and 5, normally at about pH 4.5. However, it is often more preferred to do a pre-saccharification step, lasting for about 40 to 90 minutes, at temperature of between 30-65° C., typically about 60° C., followed by complete saccharification during fermentation in a simultaneous saccharification and fermentation process (SSF).

In a fermentation process including thermal gelatinization of the granular starch the components of the antifoaming system, i.e., the lipolytic enzyme and/or the metal salt, are preferably added after the thermal gelatinization step and may preferably be added at around the same time as the yeast is added to the fermentation media. If a thermostable lipolytic enzyme is applied the components of the antifoaming system, i.e., the lipolytic enzyme and/or the metal salt, may be added before the thermal gelatinization step.

A preferred application of the antifoaming system described herein is in a raw starch hydrolysis and fermentation process, such as in a process involving treating granular starch slurry with a glucoamylase and/or alpha-amylase, and a fermenting organism, e.g., a yeast, at a temperature below the initial gelatinization temperature of granular starch. Preferably, the yeast is Ethanol Red yeast. The amylase is preferably an acid alpha-amylase, more preferably an acid fungal alpha-amylase, such as an acid fungal alpha-amylase derived from Aspergillus niger. RSH processes wherein the antifoaming system may suitably be applied are described in WO 2003/66816, WO 2003/66826, WO 2004/080923 and WO 2004/081193, all of which are hereby incorporated by reference.

In a more preferred embodiment, the raw starch hydrolysis process entails, treating granular starch slurry with a glucoamylase and/or alpha-amylase at a temperature between 0° C. and 20° C. below the initial gelatinization temperature of the granular starch, e.g., at 55° C. to 60° C., followed by treating the slurry with a glucoamylase and/or alpha amylase, yeast at a temperature of between 30° C. and 35° C.

In yet another preferred embodiment, the raw starch hydrolysis process entails the sequential steps of: (a) treating a granular starch slurry with an acid alpha-amylase and a glucoamylase at a temperature of 0° C. to 20° C. below the initial gelatinization temperature of the granular starch, e.g., at 55° C. to 60° C., preferably for a period of 5 minutes to 12 hours, such as from 5 minutes to 60 minutes, or from 5 minutes to 30 minutes, (b) treating the slurry in the presence of an acid alpha-amylase, a glucoamylase, a yeast and at least one esterase at a temperature of between 35° C. and 35° C., preferably for a period of 20 to 250 hours, e.g., for around 70 hours, to produce ethanol.

In a fermentation process which does not entail a thermal gelatinization of the granular starch, e.g., in a RSH process, the components of the antifoaming system, i.e., the lipolytic enzyme and/or the metal salt may be added at any time during the process, and most preferably at around the same time as the yeast is added to the fermentation media.

In the present disclosure the terms “slurry” and “mash” are used interchangeable in the meaning of a mixture of water and a carbohydrate source, such as plant material comprising starch and/or biomass.

### Fermenting Organism

The term “fermenting organism” refers to any organism, including bacterial and fungal organisms, including yeast and filamentous fungi, suitable for producing a desired fermentation product. Especially suitable fermenting organisms according to the invention are able to ferment, i.e., convert, sugars, glucose and/or maltose, directly or indirectly into the desired fermentation product. Examples of fermenting organisms include fungal organisms, such as yeast. Preferred yeast includes strains of the genus Saccharomyces, in particular a strain of Saccharomyces cerevisiae or Saccharomyces uvarum; a strain of Pichia, in particular Pichia stipitis, such as Pichia stipitis CBS 5773; or Pichia pastoris; a strain of the genus Candida, in particular a strain of Candida utilis, Candida diddensii, or Candida boidinii. Other contemplated yeast includes strains of Zymomonas; and Hansenula, in particular Hansenula anomala; in starin of Klyveromyces, in particular Klyveromyces fragilis; and Schizosaccharomyces, in particular Schizosaccharomyces pombe.

In one embodiment the fermenting organism is added to the fermentation medium so that the viable fermenting organism, such as yeast, count per mL of fermentation medium is in the range from 10⁵ to 10¹², preferably from 10⁷ to 10¹⁰, especially about 5×10⁷.

In ethanol production, the fermenting organism is preferably yeast, which is applied to the mash. A preferred yeast is derived from Saccharomyces spp., more preferably, from Saccharomyces cerevisiae. In preferred embodiments, yeast is applied to the mash and the fermentation is ongoing for 24-96 hours, such as typically 35-60 hours. In preferred embodiments, the temperature is generally between 26-34° C., in particular about 32° C., and the pH is generally from pH 36, preferably around pH 4-5. Yeast cells are preferably applied in amounts of 10⁵ to 10¹², preferably from 10⁷ to 10¹⁰, especially 5×10⁷ viable yeast count per ml of fermentation broth. During the ethanol producing phase the yeast cell count should preferably be in the range from 10⁷ to 10¹⁰, especially around 2×10⁸. Further guidance in respect of using yeast for fermentation can be found in, e.g., “The alcohol Textbook” (Editors K. Jacques, T. P. Lyons and D. R. Kelsall, Nottingham University Press, United Kingdom 1999), which is hereby incorporated by reference. Commercially available yeast includes, e.g., ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

### Compositions

In a preferred aspect the invention relates to a composition comprising a lipolytic enzyme and a metal salt. The composition may according to the invention be used as an antifoaming system in a fermentation process. The lipolytic enzyme is preferably selected from the group consisting of phospholipase, lysophospholipase and lipase. The metal salt is preferably selected from the group consisting of CaCl₂, CaCO₃, Ca(OH)₂, NaCl and KCl.

### Recovery

Subsequent to fermentation the fermentation product may be separated from the fermented slurry. The slurry may be distilled to extract the desired fermentation product or the desired fermentation product may be extracted from the fermented slurry by micro or membrane filtration techniques. Alternatively the fermentation product may be recovered by stripping. Methods for recovery are well known in the art.

When the fermentation product is ethanol, the ethanol, obtained according to the processes of the invention, may be used as, e.g., fuel ethanol; drinking ethanol, i.e., potable neutral spirits; or industrial ethanol.

While the antifoaming system is particularly suitable for fermentation processes it may be applied in any industrial processing of organic material in which foam develops.

### Materials and Methods

### Alpha-Amylase Activity (KNU)

The amylolytic activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.+/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

### Alpha-Amylase Activity (FAU)

The fungal alpha-amylase activity may be expressed in “Fungal Alpha-amylase Units” (FAU). One (1) FAU is the amount of enzyme which under standard conditions (i.e., at 37° C. and pH 4.7) breaks down 5260 mg solid starch (Amylum solubile, Merck) per hour. A folder AF 9.1/3, describing this FAU assay in more details, is available upon request from Novo Nordisk A/S, Denmark, which folder is hereby included by reference.

### Acid Alpha-Amylase Activity (AFAU)

Acid alpha-amylase activity may be measured in AFAU (Acid Fungal Alpha-amylase Units), which are determined relative to an enzyme standard. 1 FAU is defined as the amount of enzyme which degrades 5.260 mg starch dry matter per hour under the below mentioned standard conditions.

Acid alpha-amylase, an endo-alpha-amylase (1,4alpha-D-glucan-glucanohydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

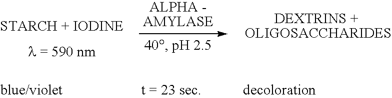

Standard Conditions/Reaction Conditions:

| | |
|---|---|
| Substrate: | Soluble starch,approx. 0.17 g/L |
| Buffer: | Citrate, approx. 0.03 M |
| Iodine (I2): | 0.03 g/L |
| CaCl₂: | 1.85 mM |
| pH: | 2.50 ± 0.05 |
| Incubation temperature: | 40° C. |
| Reaction time: | 23 seconds |
| Wavelength: | 590 nm |
| Enzyme concentration: | 0.025 AFAU/mL |
| Enzyme working range: | 0.01-0.04 AFAU/mL |

A folder EB-SM-0259.02/01 describing this analytical method in more detail is available upon request to Novozymes A/S, Denmark, which folder is hereby included by reference.

### Glucoamylase Activity (AGU)

The Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

AMG Incubation:

| | |
|---|---|
| Substrate: | maltose 23.2 mM |
| Buffer: | acetate 0.1 M |
| pH: | 4.30 ± 0.05 |
| Incubation temperature: | 37° C. ± 1 |
| Reaction time: | 5 minutes |
| Enzyme working range: | 0.5-4.0 AGU/mL |

Color Reaction:

| | |
|---|---|
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes A/S, Denmark, which folder is hereby included by reference.

### Lipolytic Activity

The lipolytic activity may be determined using tributyrine as substrate. This method is based on the hydrolysis of tributyrin by the enzyme, and the alkali consumption is registered as a function of time.

One Lipase Unit (LU) is defined as the amount of enzyme which, under standard conditions (i.e., at 30° C.; pH 7.0; with Gum Arabic as emulsifier and tributyrine as substrate) liberates 1 micromol titrable butyric acid per minute. One KLU is equal to 1000 LU.

A folder AF 95/5 describing this analytical method in more detail is available upon request to Novo Nordisk A/S, Denmark, which folder is hereby included by reference.

### EXAMPLE 1

A slurry was obtained by adding 140 kg milled wheat or barley (particle size of 50%<0.2 mm and 98%<1.0 mm and dry solids approx. 90%) to 360 liter of 65° C. water under stirring. When the temperature was 55° C. enzymes for liquefaction and saccharification was added comprising A. niger AMG (232 AGU/kg DS milled grain), A. niger acid alpha-amylase (104 AFAU/kg DS milled grain), A. oryzae alpha-amylase (58 FAU/kg DS milled grain), Bacillus licheniformis alpha-amylase (279 KNU/kg DS milled grain).

The lipolytic enzyme, a phospholipase (LECITASE ULTRA) was added in an amount of 10 KLU/kg DS milled grain and CaCl₂:2H₂O was added in an amount of 1.73 g/kg DS milled grain.

Mashing and fermentation were performed in 500 liter cylindrical stainless steel tank (height 160 cm, diameter 90 cm) fitted with a MIG-stirrer, placed 200 mm above the bottom of the tank and mantel for heating/cooling.

Mashing-in was carried out for 30 minutes under stirring at 55° C. where after the tank content was cooled to 30-32° C. before yeast pitching. Dry yeast 500 g (Danish Distillers A/S, Batch 0355 a 2006.09.11) which had been re-hydrated in 2500 mL water at 30° C., stirred lightly and allowed to stand for 15-30 minutes was pitched into the fermentor. Fermentation was performed with stirring and the temperature was held at 32° C.

The thickness of the foam layer was measured and foam volume calculated as percent of the volume of the mash at the start of the mashing. Percentage ethanol was measured by HPLC. The results are shown in table 1.

TABLE 1
Effect of antifoaming system in a raw starch ethanol process
based on milled wheat or barley.

| | | | | | | % w/w | % w/w |
|---|---|---|---|---|---|---|---|
| | | | | | | ethanol | ethanol |
| | Foam % of total | | | | | after | after |
| | mash volume, | | | | | approx. 50 | approx. 70 |
| | hour | | | | | hours | hours |
| | 2 h | 4 h | 6 h | 8 h | 12 h | 50 h | 70 h |
| Wheat | 2 | 25 | 37 | 38 | 13 | 9.2 | no data |
| Wheat | 2 | 12 | 25 | 63 | 6 | 9.3 | 9.3 |
| Wheat w. | 2 | 37 | 62 | 63 | 13 | 10.3 | 11.1 |
| Phospholipase | | | | | | | |
| Wheat w. | 2 | 19 | 12 | 6 | 1 | 8.6 | 8.8 |
| Phospholipase + CaCl₂ | | | | | | | |
| Barley w. | 2 | 37 | 62 | 63 | 38 | 9.3 | 11.1 |
| Phospholipase | | | | | | | |
| Barley w. | 1 | 2 | 3 | 3 | 3 | 10.5 | 11.1 |
| Phospholipase + CaCl₂ | | | | | | | |

1-15. (canceled)

16. A process for production of a fermentation product, which process comprises contacting a fermentation media with a fermenting organism, a lipolytic enzyme and a metal salt.

17. The process of claim 16, wherein the lipolytic enzyme is selected from the group consisting of phospholipase, lysophospholipase and lipase.

18. The process of claim 16, wherein the metal salt is selected from the group consisting of CaCl₂, CaCO₃, Ca(OH)₂, NaCl and KCl.

19. The process of claim 16, wherein the fermenting organism is a yeast.

20. The process of claim 16, wherein the fermentation product is ethanol.

21. The process of claim 16, wherein the ethanol is fuel ethanol or potable ethanol.

22. The process of claim 16, wherein fermentation is performed as part of a simultaneous saccharification and fermentation process.

23. The process of claim 16, wherein the fermentation media comprises gelatinized starch.

24. The process of claim 16, wherein the fermentation media comprises ungelatinized starch.

25. The process of claim 16, wherein the fermentation step is carried out in the presence a glucoamylase and/or an amylase.

26. The process of claim 16, wherein the fermentation media comprises a starch material derived from a plant selected from the group consisting of corn, wheat, barley, and milo.

27. The process of claim 16, further comprising contacting the fermenting microorganism or the fermentation media with an enzyme selected from the group consisting of protease, phytase, and cellulase.

28. A composition comprising a lipolytic enzyme and a metal salt.

29. An antifoaming system comprising a lipolytic enzyme and a metal salt.
